(19) United States
(12) Patent Application Publication
(10) Pub. No.: US 2008/0161777 A1
(43) Pub. Date: Jul. 3, 2008

(54) Shaped Cardiac Introducers for Ventricular Septal and Right Ventricular Apical Access and Method of Using Same

(75) Inventors: Paul A. Kurth, Santa Barbara, CA (US); Seth J. Worley, Lancaster, PA (US)

Correspondence Address: MYERS DAWES ANDRAS & SHERMAN, LLP, 19900 MACARTHUR BLVD., SUITE 1150, IRVINE, CA, 92612, US

(73) Assignee: Pressure Products Medical Supplies, Inc., Santa Barbara, CA (US)

(21) Appl. No.: 11/814,857

(22) PCT Filed: Jan. 24, 2006
(86) PCT No.: PCT/US06/02460

§ 371 Date: Jul. 26, 2007

Related U.S. Application Data
(60) Provisional application No. 60/647,857, filed on Jan. 27, 2005.

(51) Int. Cl. A61M 25/01

(52) U.S. Cl. 604/532

(57) ABSTRACT

A class of introducers is comprised of a plurality of straight sections and at least two curved sections with a pre-determined sense of curvature preceded, separated and followed by the straight sections, which introducer is adapted by its shape to access the septal wall or apex of a heart. One of the plurality of straight sections is a distal-most straight section. The straight and curved sections define a predetermined three dimensional shape by means of the distal straight section lying out of plane with respect to the remaining curved and straight sections.

### RELATED APPLICATIONS

The present application is related to U.S. Provisional Patent Application Ser. No. 60/647,857, filed on Jan. 27, 2005, which is incorporated herein by reference and to which priority is claimed pursuant to 35 USC 119

### BACKGROUND OF THE INVENTION

1. Field of the Invention

The invention relates to the field of cardiac introducers for access to the apex of the right ventricle and to the ventricular septal wall.

2. Description of the Prior Art

Pacing leads are typically implanted into the right heart chambers and particularly into the apical area and/or the septal wall of the right ventricle. While introduction of an introducer into the right ventricle is usually easily accomplished, it is not enough just to be in the right ventricle, but it is desirable to have the distal tip of the introducer positioned in that part of the right ventricle with respect to the ventricular tissue that is the target for implantation. While steering introducers or catheters are well known, the time and effort spent in steering an introducer to the target is time and effort, which may not always be well spent.

In certain anatomical variations of the heart and certain lead characteristics make the placement of the pacemaker lead in the apex of the right ventricle and right ventricular wall very difficult. Therefore, some kind of conduit is needed whereby such placements can be reliably and quickly made in these circumstances.

Successful access to a desired location in the heart by means of an intravascular catheter is often the margin between success and failure in a cardiac intervention. Further, the quicker access can be reliably achieved, the better the chance of a good outcome for the patient and the less the trauma associated with the intervention.

### BRIEF SUMMARY OF THE INVENTION

The illustrated embodiment of the invention is a class of introducers comprised of: a plurality of straight sections; and at least two curved sections with a predetermined sense of curvature preceded, separated and followed by the straight sections, which introducer is adapted by its shape to access the septal wall or apex of a heart.

One of the plurality of straight sections is a distal-most straight section. The straight and curved sections define a predetermined three dimensional shape by means of the distal straight section lying out of plane with respect to the remaining curved and straight sections.

The plurality of straight sections and two curved sections comprise a proximal straight section, a first curved section R1 having a curvature with the predetermined sense of curvature and contiguous with the initial straight section, a first straight section S1 contiguous with the first curved section R1, a second curved section R2 having the predetermined sense of positive curvature and contiguous with the first straight section S1, a second straight section S2 contiguous with the second curved section R2. The predetermined sense of curvature in the illustration is determined by the view and is shown as counterclockwise curvature when moving from the proximal end toward the distal end of the introducer, i.e. curving to the left.

In one embodiment the curvature of the first curved section R1 has a radius of approximately 1-3 inches, preferably approximately 2.0 inches, and a length of approximately 1-3 inches, preferably approximately 2.3 inches, where the first straight section S1 has length of approximately 0 to 3.0 inches, preferably approximately 1.1 inches, where the curvature of the second curved section R2 has a radius of approximately 2-4 inches, preferably approximately 2.9 inches, and a length of approximately 2.5-4.5 inches, preferably approximately 3.4 inches, and where the second straight section S2 has length of approximately 2-6 inches, preferably approximately 4.5 inches. The proximal straight section has a length of approximately 5-11 inches, preferably approximately 7.4 inches.

In another characterization of the invention one of the plurality of straight sections is a proximal straight section and one is a distal-most straight section and where the introducer has a length of approximately 13-23 inches preferably approximately 18.6 inches, and collectively curves rearwardly to incline the distal-most straight section relative to the proximal straight section by an interior angle of approximately 30° to 90°, preferably approximately 50°.

In the illustrated embodiment the proximal straight section, the first curved section R1, the first straight section S1, and the second curved section R2 lie in a first common plane, and where at least a portion of the second straight section S2 lies in a second dihedral plane. More particularly, the second dihedral plane inclined with respect to the common plane by approximately 20°-40°, preferably approximately 30°.

The illustrated embodiment of the introducer further comprises a third curved section R3 having a sense of curvature opposite to the predetermined sense of curvature and disposed between the second curved section R2 and second straight section S2. For example, the curvature of the first curved section R1 has a radius of approximately 1-3 inches, preferably approximately 2.1 inches, and a length of approximately 1-3.5 inches, approximately preferably 2.3 inches, where the first straight section S1 has length of approximately 0-3 inches, preferably approximately 1.1 inches, where the curvature of the second curved section R2 has a radius of approximately 1-4 inches, preferably approximately 2.6 inches, and a length of approximately 0.5-3.5 inches, preferably approximately 1.8 inches, where the curvature of the third curved section R3 has a radius of approximately 2.5-4.5 inches, preferably approximately 3.6 inches, and a length of approximately 1-3 inches, preferably 2.2 inches, and where the second straight section S2 has length of approximately 0-4 inches, preferably approximately 2.0 inches. The proximal straight section has a length of approximately 5-11 inches, preferably approximately 7.4 inches.

Characterized in another way the embodiment with three curves has one of the plurality of straight sections as a proximal straight section and one as a distal-most straight section. The introducer has a length of approximately 13-23 inches, preferably approximately 16.8 inches, and collectively curves outwardly to incline the distal-most straight section relative to the proximal straight section by an interior angle of approximately 80°-130°, preferably approximately 111°.

Similarly, the proximal straight section, the first curved section R1, the first straight section S1, the second curved section R2 and the third curved section R3 lie in a first common plane, and where at least a portion of the second straight section S2 lies in a second dihedral plane. The second dihedral plane inclined with respect to the common plane by approximately 20°-40°, preferably approximately 30°.

The invention thus can also be characterized in one embodiment as an introducer comprising an introducer body prebiased to assume a shape having at least three curved sections of which one pair has opposite curvature to each other, and which curved sections of opposite curvature are contiguous with each other, the other curved section being noncontiguous with the pair, the noncontiguous curved section and the pair of contiguous curve sections being preceded, separated and followed by straight sections, which introducer is particularly adapted to allow access to the apex of the heart. The sections define a predetermined three dimensional shape by means of the distal curved and straight sections lying out of plane with respect to the remaining curved and straight sections. The body comprises an initial straight section, a first curved section R1 having a curvature with a positive curvature and contiguous with the initial straight section, a first straight section S1 contiguous with the first curved section R1, a second curved section R2 having a positive curvature and contiguous with the first straight section S1, a third curved section R3 having a negative curvature and contiguous with the second curved section R2 to form the pair of curved sections, a second straight section S2 contiguous with the third curved section R3.

Still further an embodiment of the invention can be defined as an introducer consisting only of a plurality of straight sections and two curved sections with a predetermined sense of curvature preceded, separated and followed by the straight sections, which introducer is adapted by its shape to access the septal wall of a heart.

Still further yet an embodiment of the invention can be defined as introducer consisting only of a plurality of straight sections and three curved sections with a predetermined sense of curvature preceded, separated and followed by the straight sections, which introducer is adapted by its shape to access the apex of a heart.

While the apparatus and method has or will be described for the sake of grammatical fluidity with functional explanations, it is to be expressly understood that the claims, unless expressly formulated under 35 USC 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 USC 112 are to be accorded full statutory equivalents under 35 USC 112. The invention can be better visualized by turning now to the following drawings wherein like elements are referenced by like numerals.

### BRIEF DESCRIPTION OF THE DRAWINGS

FIG. 1 is a plan view of a first embodiment of the invention.

FIG. 2 is the plan view of the first embodiment of the invention relabeled to more simply identify its shaped sections.

FIG. 3 is a perspective view of the embodiment of FIGS. 1 and 2 wherein the introducer includes out-of-plane sections.

FIG. 4 is a plan view of a second embodiment of the invention.

FIG. 5 is the plan view of the embodiment of FIG. 4 relabeled to more simply identify its shaped sections is a plan view of a fourth embodiment of the invention.

FIG. 6 is a perspective view of the embodiment of FIGS. 4 and 5 wherein the introducer includes out-of-plane sections.

The invention and its various embodiments can now be better understood by turning to the following detailed description of the preferred embodiments which are presented as illustrated examples of the invention defined in the claims. It is expressly understood that the invention as defined by the claims may be broader than the illustrated embodiments described below.

### DETAILED DESCRIPTION OF THE PREFERRED EMBODIMENTS

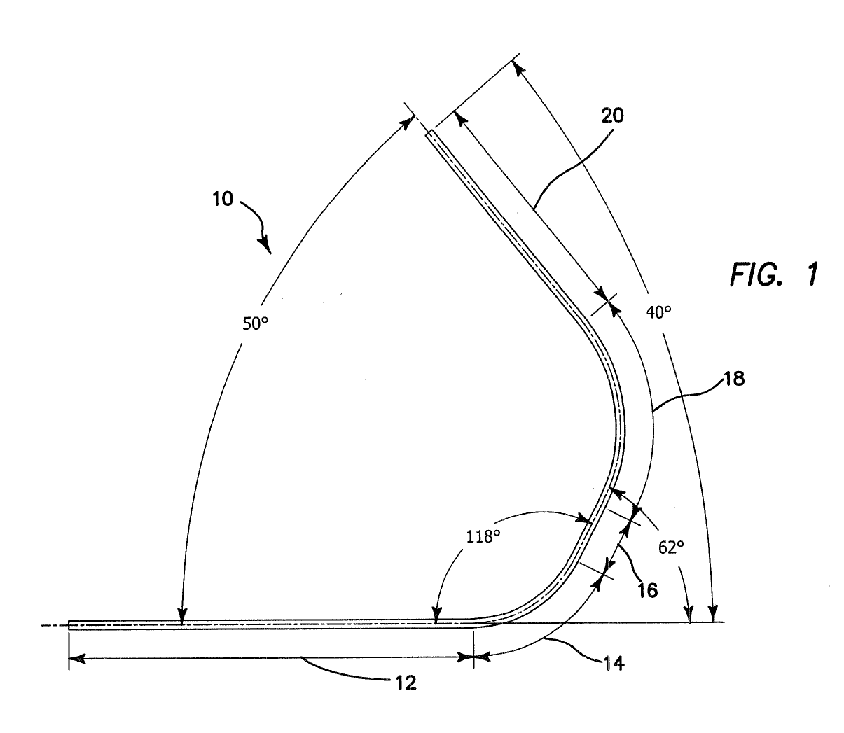

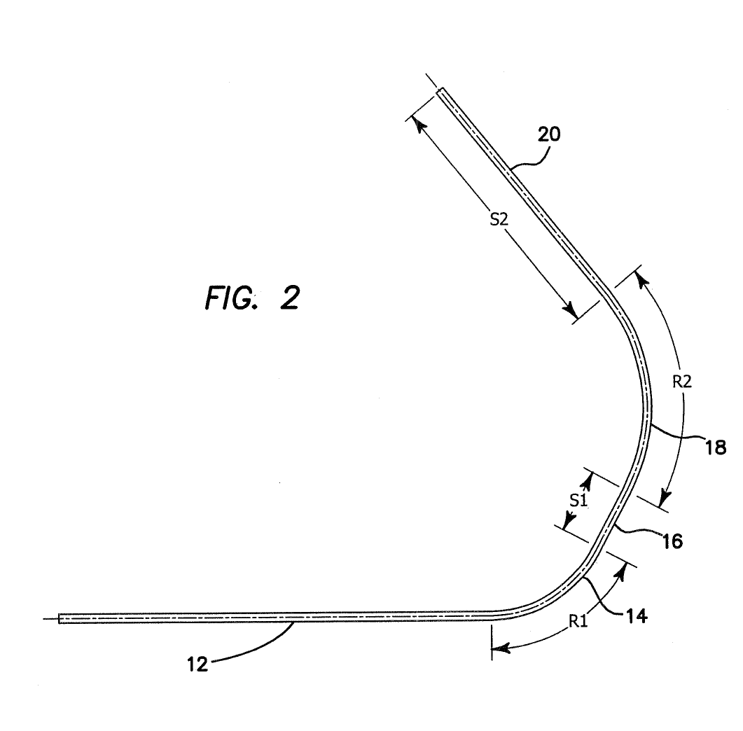

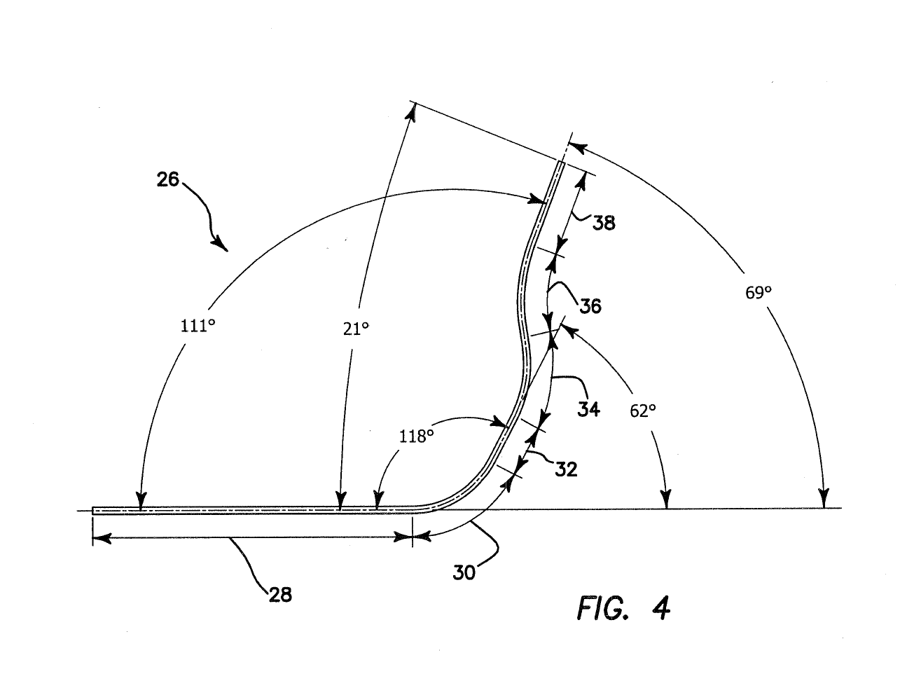

FIGS. 1-3 are embodiments used for implanting intraventricular septal pacing leads and FIGS. 4-6 are embodiments used for implanting right ventricular apex pacing leads. FIGS. 1 and 2 are plan views of a first embodiment of a shaped of a septal wall introducer 10. Introducer 10 is particularly adapted to provide access to the interventricular muscular septal wall for pacing by access through the right ventricle and right atrium. The body section 12 of introducer 10 is an unbiased flexible, generally straight section which extends through the vascular system and exterior to the body. Body section 12 smoothly matches first curved section 14, R1, which has a first radius of curvature of 2.08±1.04 inches on a center to the left of introducer 10 as seen in FIGS. 1 and 2, and an arc length of 2.26±1.13 inches. First section 14 smoothly continues into straight section 16, S1, which has a length of 1.07 inch±0.53. The angle between straight sections 12 and 16 when laid flatly and when otherwise left to assume its relaxed shape is approximately 118°.

Straight section 16 smoothly continues into second curved section 18, R2, which has a radius of curvature of 2.92 inch±1.46 on a center to the left of introducer 10 as seen in FIGS. 1, and 2, and an arc length of 3.44±1.72 inches. A radius with a center left of the introducer in FIGS. 1, 2, 4 and 5 will be defined in this specification as a positive curvature, where a radius with a center to the right of the introducer will be defined as a negative curvature. Introducer 10 then terminates with a second straight section 20, S2, which has a length of 4.47±2.23 inches.

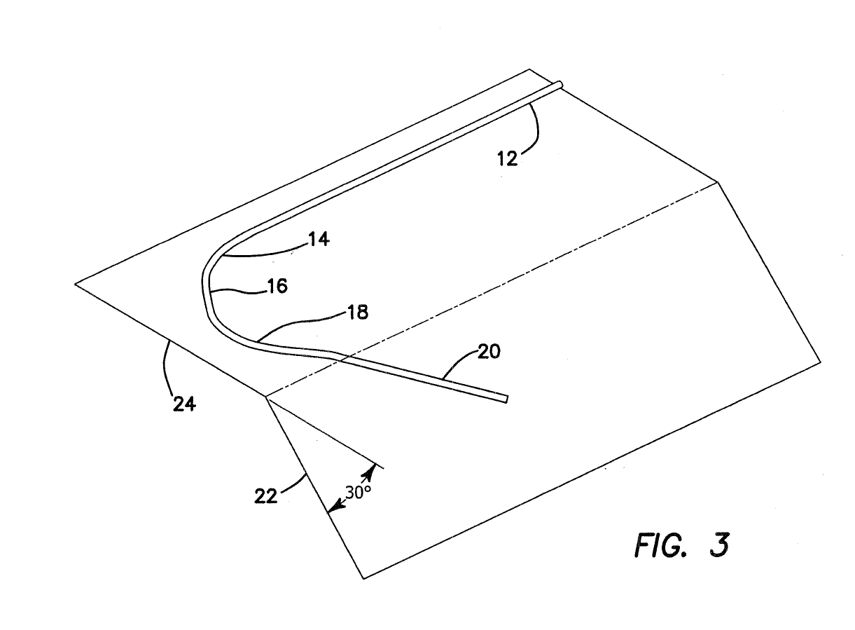

The angle between straight sections 12 and 20 when laid on the relaxed dihedral planes 22 and 24 as shown in FIG. 3 is approximately 40°. FIG. 3 shows sections 12-18 as commonly lying when relaxed in a common plane 42 and then with straight section 20 curving out of plane on to inclined plane 22 which is inclined by a dihedral angle of approximately 30° with respect to plane 24.

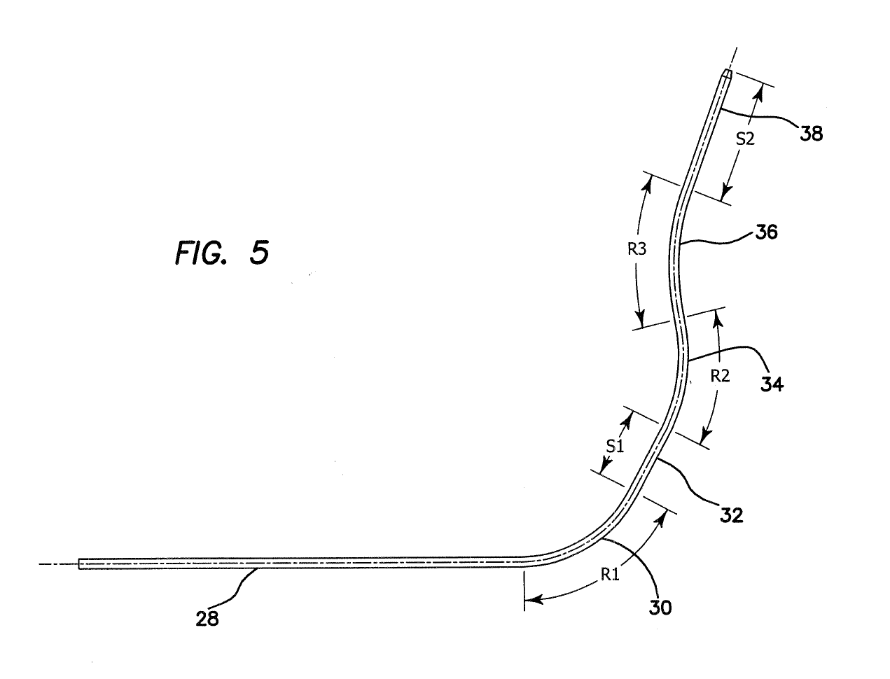

FIGS. 4 and 5 are plan views of an embodiment of a shape introducer 26 adapted to access the apex or apical areas of the heart. Introducer 26 is particularly adapted to provide access to the apex of the right ventricle of the heart by access through the right atrium and tricuspid valve for medical device deployment. The body section 28 of introducer 26 is an unbiased flexible, generally straight section which extends through the vascular system and exterior to the body. Body section 28 smoothly matches first curved section 30, R1′, which has a first radius of curvature of 2.06±1.03 inches on a center to the left of introducer 26 as seen in FIGS. 4 and 5, and an arc length of 2.26±1.13 inches. First curved section 30 smoothly continues into straight section 32, S1′, which has a length of 1.08 inch±0.54. The angle between straight sections 28 and 32 when laid flatly and when otherwise left to assume its relaxed shape is approximately 118°.

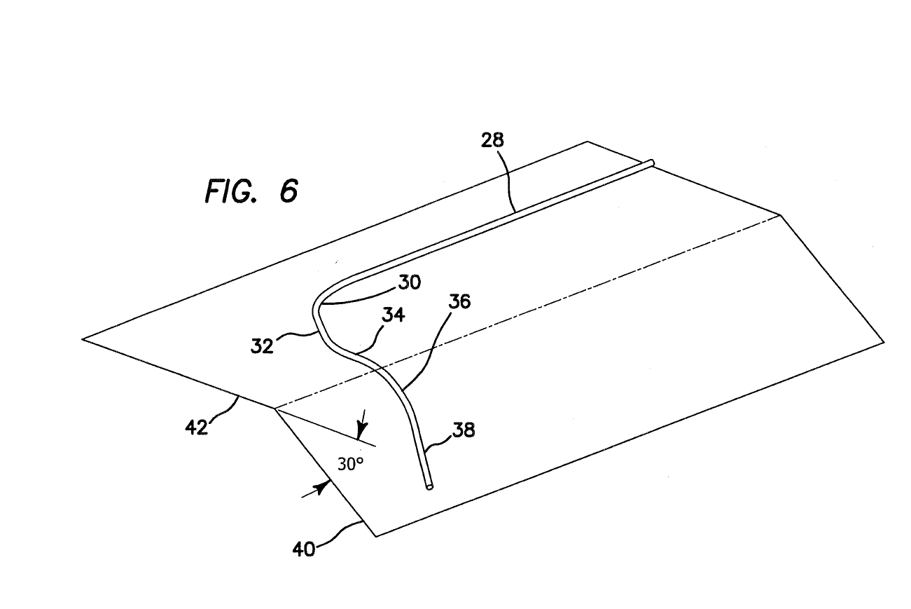

Straight section 32 smoothly continues into second curved section 34, R2, which has a length of 2.58 inch±1.29 on a center to the left of introducer 26 as seen in FIGS. 4 and 5, and an arc length of 1.83±0.91 inches. Curved section 34 smoothly continues into third curved section 36, R3, which has a length of 3.58 inch±1.79 on a center to the right of introducer 26 as seen in FIGS. 4 and 5, and an arc length of 2.16±1.08 inches. Introducer 26 then terminates with a second straight section 38, S2, which has a length of 2.01±1.00 inches. The angle between straight sections 28 and 38 when laid on the adjacent dihedral planes 40 and 42 as shown in FIG. 6 is approximately 69°. FIG. 6 shows sections 28-34 as commonly lying when relaxed in a common plane 42 and then with curved section 36 and straight section 38 curving out of plane on to inclined plane 40 which is inclined by a dihedral angle of approximately 30° with respect to plane 42.

Many alterations and modifications may be made by those having ordinary skill in the art without departing from the spirit and scope of the invention. Therefore, it must be understood that the illustrated embodiment has been set forth only for the purposes of example and that it should not be taken as limiting the invention as defined by the following invention and its various embodiments.

For example, the above embodiments have been shown with exemplary dimensions to provide an example of an enabled shape, but it is expressly contemplated that the ranges and dimensions could be varied from those given, while the introducer has the same essential geometry which enables it to access the apex or septal wall of the heart by following the spirit of the invention. Thus, not only scaling of the disclosed shapes is included within the scope of the invention, but also modifications of shape which provide substantially similar tracking through the vascular system to place the distal end of the introducer in the same region of the heart. Also included within the scope of the invention are modifications of the disclosed shape to accommodate changes in the anatomy of the heart, so that an introducer whose shape has been modified in a manner consistent with the invention, but adapted to the anatomies of juvenile hearts and myopathic hearts, is expressly contemplated.

Therefore, it must be understood that the illustrated embodiment has been set forth only for the purposes of example and that it should not be taken as limiting the invention as defined by the following claims. For example, notwithstanding the fact that the elements of a claim are set forth below in a certain combination, it must be expressly understood that the invention includes other combinations of fewer, more or different elements, which are disclosed in above even when not initially claimed in such combinations. A teaching that two elements are combined in a claimed combination is further to be understood as also allowing for a claimed combination in which the two elements are not combined with each other, but may be used alone or combined in other combinations. The excision of any disclosed element of the invention is explicitly contemplated as within the scope of the invention.

The words used in this specification to describe the invention and its various embodiments are to be understood not only in the sense of their commonly defined meanings, but to include by special definition in this specification structure, material or acts beyond the scope of the commonly defined meanings. Thus if an element can be understood in the context of this specification as including more than one meaning, then its use in a claim must be understood as being generic to all possible meanings supported by the specification and by the word itself.

The definitions of the words or elements of the following claims are, therefore, defined in this specification to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a subcombination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptionally equivalent, what can be obviously substituted and also what essentially incorporates the essential idea of the invention.

We claim:

1. An introducer comprising:
a plurality of straight sections; and
at least two curved sections with a predetermined sense of curvature preceded, separated and followed by the straight sections, which introducer is adapted by its shape to access the septal wall or apex of a heart.

2. The introducer of claim 1 where one of the plurality of straight sections is a distal-most straight section and where the straight and curved sections define a predetermined three dimensional shape by means of the distal straight section lying out of plane with respect to the remaining curved and straight sections.

3. The introducer of claim 1 where the plurality of straight sections and two curved sections comprise a proximal straight section, a first curved section R1 having a curvature with the predetermined sense of curvature and contiguous with the initial straight section, a first straight section S1 contiguous with the first curved section R1, a second curved section R2 having the predetermined sense of positive curvature and contiguous with the first straight section S1, a second straight section S2 contiguous with the second curved section R2.

4. The introducer of claim 3 where the curvature of the first curved section R1 has a radius of approximately 1 to 3 inches and a length of approximately 1-3 inches, where the first straight section S1 has length of approximately 0 to 3 inches, where the curvature of the second curved section R2 has a radius of approximately 2-4 inches and a length of approximately 2.5 to 4.5 inches, and where the second straight section S2 has length of approximately 2-6 inches.

5. The introducer of claim 3 where proximal straight section has a length of approximately 5-11 inches.

6. The introducer of claim 1 where one of the plurality of straight sections is a proximal straight section and one is a distal-most straight section and where the introducer has a length of approximately 13-23 inches and collectively curves rearwardly to incline the distal-most straight section relative to the proximal straight section by an interior angle of approximately 30° to 90°.

7. The introducer of claim 3 where the proximal straight section, the first curved section R1, the first straight section S1, and the second curved section R2 lie in a first common plane, and where at least a portion of the second straight section S2 lies in a second dihedral plane.

8. The introducer of claim 7 where the second dihedral plane inclined with respect to the common plane by approximately 20°-40°.

9. The introducer of claim 6 where the proximal straight section, the first curved section R1, the first straight section S1, and the second curved section R2 lie in a first common plane, and where at least a portion of the second straight section S2 lies in a second dihedral plane and where the second dihedral plane inclined with respect to the common plane by approximately 20°-40°.

10. The introducer of claim 3 further comprising a third curved section R3 having a sense of curvature opposite to the predetermined sense of curvature and disposed between the second curved section R2 and second straight section S2.

11. The introducer of claim 10 where the curvature of the first curved section R1 has a radius of approximately 1-3 inches and a length of approximately 1-3 inches, where the first straight section S1 has length of approximately 1-3.5 inches, where the curvature of the second curved section R2 has a radius of approximately 1-4 inches and a length of approximately 0.5-3.5 inches, where the curvature of the third curved section R3 has a radius of approximately 2.5-4.5 inches and a length of approximately 1-3 inches, and where the second straight section S2 has length of approximately 0-4 inches.

12. The introducer of claim 11 where proximal straight section has a length of approximately 5-11 inches.

13. The introducer of claim 10 where one of the plurality of straight sections is a proximal straight section and one is a distal-most straight section and where the introducer has a length of approximately 13-23 inches and collectively curves outwardly to incline the distal-most straight section relative to the proximal straight section by an interior angle of approximately 80°-130°.

14. The introducer of claim 10 where the proximal straight section, the first curved section R1, the first straight section S1, the second curved section R2 and the third curved section R3 lie in a first common plane, and where at least a portion of the second straight section S2 lies in a second dihedral plane.

15. The introducer of claim 14 where the second dihedral plane inclined with respect to the common plane by approximately 20°-40°.

16. An introducer comprising an introducer body prebiased to assume a shape having at least three curved sections of which one pair has opposite curvature to each other, and which curved sections of opposite curvature are contiguous with each other, the other curved section being noncontiguous with the pair, the noncontiguous curved section and the pair of contiguous curve sections being preceded, separated and followed by straight sections, which introducer is particularly adapted to allow access to the apex of the heart.

17. The introducer of claim 16 where the sections define a predetermined three dimensional shape by means of the distal curved and straight sections lying out of plane with respect to the remaining curved and straight sections.

18. The introducer of claim 16 where the body comprises an initial straight section, a first curved section R1 having a curvature with a positive curvature and contiguous with the initial straight section, a first straight section S1 contiguous with the first curved section R1, a second curved section R2 having a positive curvature and contiguous with the first straight section S1, a third curved section R3 having a negative curvature and contiguous with the second curved section R2 to form the pair of curved sections, a second straight section S2 contiguous with the third curved section R3.

19. An introducer consisting of:
a plurality of straight sections; and
two curved sections with a predetermined sense of curvature preceded, separated and followed by the straight sections, which introducer is adapted by its shape to access the septal wall of a heart.

20. An introducer consisting of:
a plurality of straight sections; and
three curved sections with a predetermined sense of curvature preceded, separated and followed by the straight sections, which introducer is adapted by its shape to access the apex of a heart.
